(19) United States
(12) Patent Application Publication
(10) Pub. No.: US 2009/0254142 A1
(43) Pub. Date: Oct. 8, 2009

(54) Treating Medical Conditions of Hollow Organs

(75) Inventors: Stuart D. Edwards, Salinas, CA (US); Peter H. Muller, Woodside, CA (US); Thomas C. Wehman, Cupertino, CA (US)

Correspondence Address: Soodabek Tronson, 405 El Camino Real, Menlo Park, CA, 94025, US

(73) Assignee: SILHOUETTE MEDICAL, USA, Prunedale, CA (US)

(21) Appl. No.: 12/108,499

(22) Filed: Apr. 23, 2008

Related U.S. Application Data
(63) Continuation-in-part of application No. 12/099,349, filed on Apr. 8, 2008, now abandoned.

(51) Int. Cl. A61N 1/04

(52) U.S. Cl. 607/40

(57) ABSTRACT

A minimally invasive treatment and appartus for treating medical conditions of hollow organs is described. Electrodes positioned within the organ and in surface contact with the underlying glands, nerves, and muscle walls of the organ apply energy to specific glandular, nerve, or muscular areas to alter the organ's operation.

### FIELD OF THE INVENTION

This patent application concerns treating medical conditions of hollow organs. As but an example, the application describes treating the hollow organs of the digestive system to treat body-weight related conditions.

All patents and published patent applications referred to herein are incorporated by reference into this patent application in their entirety.

### BACKGROUND

The human body has several organs that are considered hollow. For example, the following organs are considered hollow: organs of the GI Tract (esophagus, stomach, small and large intestines), bladder, ear canal, nasal sinuses, female reproductive system (vagina, vaginal canal, uterus, fallopian tubes) and the lungs. This list is exemplary and not all inclusive.

Each of these and other hollow organs can be subject to medical conditions such as cancer or conditions resulting from loosening of the muscles underderlying the organ. Treatment for these medical conditions range from pharmaceutical therapies to highly invasive surgeries.

As an example, obesity is one major medical condition that affects several hollow organs of the GI Tract. Obesity is directly associated with other medical disorders, such as: osteoarthritis (especially in the hips), sciatica, varicose veins, thromboembolism, ventral and hiatal hernias, hypertension, insulin resistance and hyperinsulinemia; premature death; type 2 diabetes; heart disease; stroke; hypertension; gall bladder disease; GI tract cancers; incontinence; psychological disorders; sleep apnea; gastro esophageal reflux disease (GERD); and liver disease. Reducing obesity, reduces the effects of these conditions, provided the weight loss is significant and enduring. This, of course, is the challenge to the patient and practitioner.

Current obesity treatments include behavior modification, pharmaceutical interventions, and invasive surgeries.

One problem with behavior modification is patient compliance. Significant and maintained weight loss demand enormous levels of patient compliance over a long time.

Problems with pharmaceutical intervention include drug dependence and adverse side effects. Amphetamine analog treatments involve habitual use of addictive drugs to produce and maintain significant weight loss. Dexfenfluramine and fenfluramine treatments often result in primary pulmonary hypertension and cardiac valve abnormalities. Drugs such as sibutramine substantially increase blood pressure in many patients.

Surgical obesity treatments include invasive surgical procedures such as: gastric banding, bariatric surgery, and liposuction. While current surgical procedures can be effective, the overall rates of surgical mortality and associated hepatic dysfunction are so high that surgical treatments are only indicated for younger patients who are morbidly obese.

The following table outlines various conventional treatments for obesity and their associated costs and issues.

| Treatment | Cost | Issues |
|---|---|---|
| Diet, Exercise | Minimal | 90% unsuccessful |
| and Behavior | | |
| Modification | | |
| Pharmacotherapy | $2,200-$3,000/year | Moderate benefits and risk of |
| | | dependence |
| Very Low Calorie | $3,300-$4,000 + | Patients of regain weight |
| Diet and | Testing & Monitoring | |
| Medically | | |
| Supervised | | |
| Programs | | |
| Gastric Banding | $15,000-$25,000 | Invasive, risks, complications, |
| | | long-term care, costly |
| Bariatric Surgery | $25,000 + | Invasive, risks, complications, |
| | | long-term care, costly |
| Liposuction | $5,000-$7,500 | Invasive, risks, complications, |
| | | long-term care, costly |

U.S. Pat. No. 7,326,207 proposes treating obesity by mapping (for example, using a visualization apparatus, such as but not limited to endoscopes or fluoroscopes) and ablating nerves in targeted stomach areas by creating patterns of thermal lesions. The nerves are ablated using surface electrodes that penetrate the nerves during energy application. Mapping is required to properly position the electrodes where they can penetrate the nerves. Physiological changes caused by tissue ablation create a sense of satiety in the patient by directly modulating nerves responsible for hunger sensation or by modulating the nerves inhibiting the let-down reflex of the stomach muscles that are digestion precursors.

Despite the treatment described in the '207 patent, there is room for further improvement in the field of obesity treatment and the treatment of other medical conditions that affect hollow organs.

### BRIEF DESCRIPTION OF THE DRAWINGS

FIGS. 1A and 1B are simplified depictions of a mammalian digestive system.

FIG. 2A shows an exemplary stomach treating portion of an apparatus for treating body-weight related conditions.

FIG. 2B shows a schematic of an exemplary external control unit for use with the stomach treating portion of FIG. 2A.

FIG. 3A-3D depict different exemplary embodiments of a balloon for expanding the stomach.

FIG. 4A-L show various steps of the therapeutic procedure.

FIG. 5 shows the profile of a treated stomach about 8-12 weeks, post-op.

FIGS. 6A, 6B are graphs summarizing animal testing results.

FIG. 7 is a graph estimating the effects of the therapy on humans.

### DETAILED DESCRIPTION OF THE PREFERRED EMBODIMENTS

### Introduction and Summary

As previously mentioned, this patent application concerns treating medical conditions of hollow organs. Most generally, this patent application proposes applying energy to the muscles or glands underlying the hollow organs to alter the muscular profile of the organ and/or its biomechanical operation and/or using surface contacts that can be easily positioned to directly apply their energy to the surfaces of nerve branches associated with the communication between the hollow organ and the brain. To make sure the nerves, muscles, or glands are exposed to the surface contacts that provide the energy, an organ expander expands the organ beyond its normal volume until organ mucosa is separated and underlying nerves, muscles, and glands are exposed. The organ expander may be in the form of a balloon. These balloons do more than prior art balloons that merely expand to a point where the balloon is firmly positioned in a volume fairly close to the organ's normal volume. At a minimum, this application proposes that when the surface contacts directly apply their energy to the underlying nerves, muscles or glands, nerve signal transmission is modified, muscle profile is changed to a profile more suitable for reaching treatment goals, or the gland's enzyme release reduced.

### Anatomical Background

As an example of the application of the teachings of this application, the hollow organ will comprise any of the hollow organs of the digestive system. As a further example, the hollow organ of the digestive system will be the stomach.

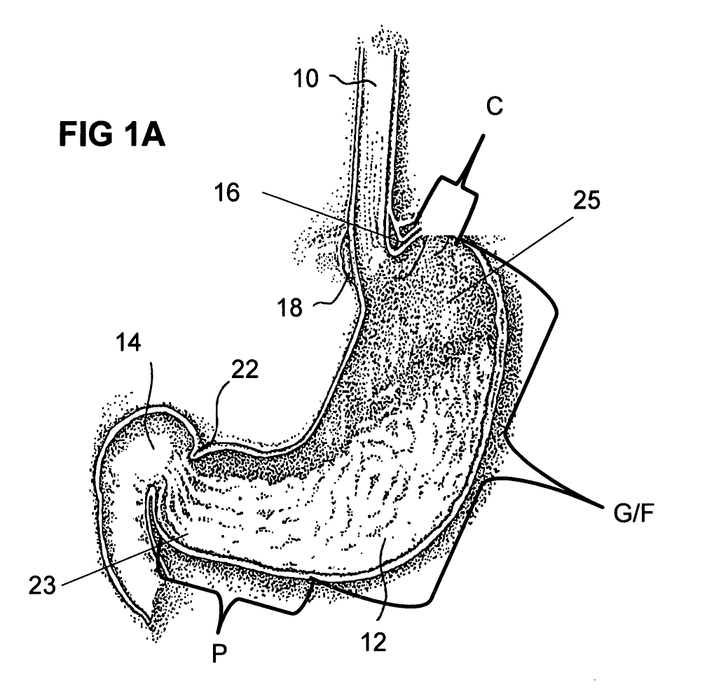

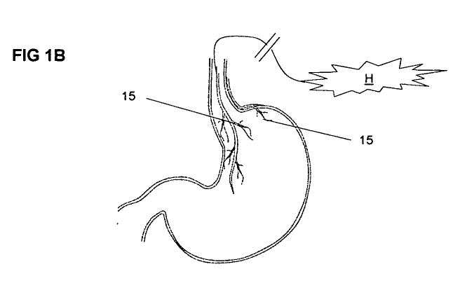

In particular, FIGS. 1A and 1B are simplified depictions of a mammalian digestive system. These FIGS are not intended to be strictly accurate in an anatomic sense or imply that the teachings of this patent application are limited strictly to treating the digestive system. The drawings show the digestive system in somewhat diagrammatic form for purposes of discussion.

In FIG. 1A, esophagus 10, a muscular tube, carries food from the mouth to the stomach 12. Wavelike contractions in the muscles in the walls of the esophagus 10 move food to the stomach 12. The interior esophagus walls include glands that secrete mucus, which aids moving food by acting as lubrication.

The stomach 12, located in the upper left hand side of the abdomen, lays between the esophagus 10 and the small intestine 14. In people and most animals, the stomach 12 is a simple baglike organ.

FIG. 1B depicts branches 15 of the vagal nerve that connect stomach 12 with the hindbrain H. Hindbrain H is believed to be the neurological source for the hunger sensation.

The volume of an average adult stomach is a little over one quart (−0.95 liter). The stomach 12 stores and digests food. Pyloric sphincter 22, distal of pylorus 23, surrounds and controls the size of the duodenal opening between stomach 12 and small intestine 14. The pyloric sphincter 22 allows liquefied food in the stomach 12 to flow slowly into the intestines 14. The pyloric sphincter 22 keeps non-liquid food in stomach 12 until the food is processed into a more flowable, liquid form. The time food spends in stomach 12 varies. Usually, stomach 12 empties in three to five hours.

The upper end of stomach 12 connects with the esophagus 10 at cardiac notch 16. The muscular ring called the lower esophageal sphincter 18 surrounds the opening between the esophagus 10 and the stomach 12. The funnel-shaped region of the stomach 12 immediately next to sphincter 18 is the cardia 20. Positioned below the cardia 20 is the fundus 25 of the stomach. Using these anatomical features as landmarks or guides, the human stomach is often described as having three zones, namely: cardiac zone, gastric/fundic zone, and pyloric zone. This application focuses on treating body-weight related conditions by using contact electrodes for directly applying energy in the vicinities of either:

(1) nerve tissue that allow nerve pulse communication between the hindbrain H and stomach 12; and/or

(2) stomach tissue to ablate tissue in one or more areas where food is either processed or absorbed by the body, for example, the cardiac, gastric/fundic, and pyloric zones.

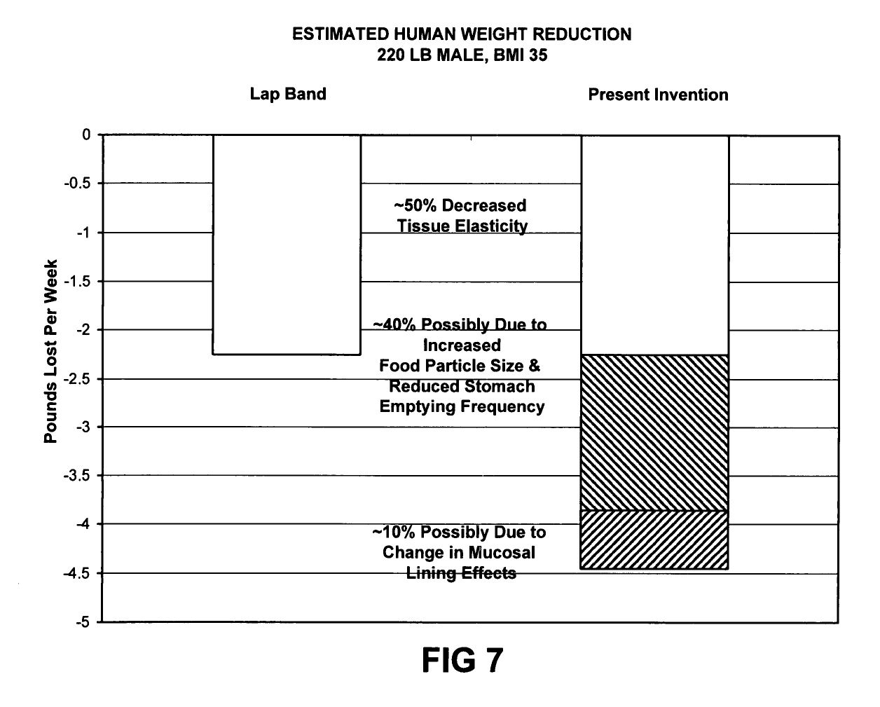

Additionally, treatment may be expanded to other areas, such as the small intestine (and associated nerves), where about 95% of all food absorption occurs. Ablation, or causing cell death, produces lesions. If the lesions are large enough, they evoke tissue-healing and intervention of fibroblasts, myofibroblasts, macrophages, and other cells. Healing results in tissue contraction (shrinkage), decreased volume and altered biomechanical properties. In contrast with other treatments for, e.g., obesity, the current application and method do more than merely try to prolong patient satiety. The current application also describes directly affecting the digestive process to reduce food absorption. Ablation of cells in the cardiac, gastric/fundic, and pyloric zones should treat weight-related conditions and reduce a patient's body weight for the following reasons (see also FIG. 7):

CARDIAC AND FUNDIC-GASTRIC ZONES—The cardia contains the cardiac glands (not shown) and the fundic-gastric zone contains the fundic glands (not shown). The cardiac and fundic glands release digestive enzymes (ghrelin, pepsin and rennin) and HCl which are all used during digestion to break down food. Ablating a portion of the cardiac and gastric-fundic zones, e.g., the cardiac and fundic glands, therefore, reduces the release of ghrelin, pepsin, rennin and HCl, thereby reducing the amount of food digested by the body. Therefore, more food particles would pass through the patient's body undigested.

PYLORIC ZONE—The pyloric sphincter controls food flow out of the stomach (emptying cycle) as well as the size of the food particle that may flow out of the stomach. The wider the sphincter may open, the larger the food particle that may flow out. Ablating pyloric muscle tissue decreases the size of the pyloric opening and the size of food particles that may flow out, thereby lengthening the emptying cycle (longer sensation of satiety).

The gastric zone also includes the lesser curvature of the stomach, which contains nerves that control peristalsis of the stomach walls. Peristalsis contributes to digestion by physically reducing the size of food particles in the stomach. Ablating portions of the muscles of the lesser curvature reduces peristalsis and increase food particle size. These larger food particles, when passed through the pyloric sphincter, cannot be digested through the small intestine and therefore would pass through the patient's body undigested. Finally, ablating gastric zone tissue may also affect the gastric glands and reduce HCl production in the stomach even more (see above).

Against this anatomical and physiological background, an exemplary apparatus and method for treating body-weight related medical conditions associated with hollow organs will be described.

### Treatment Apparatus

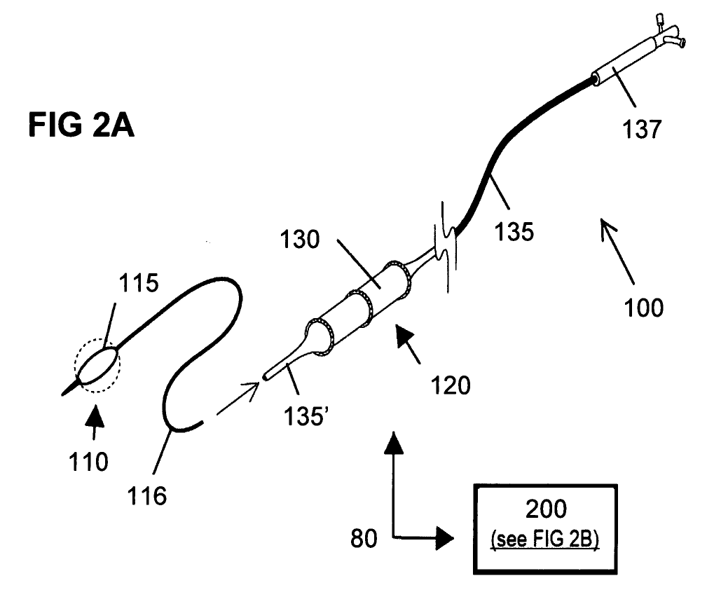

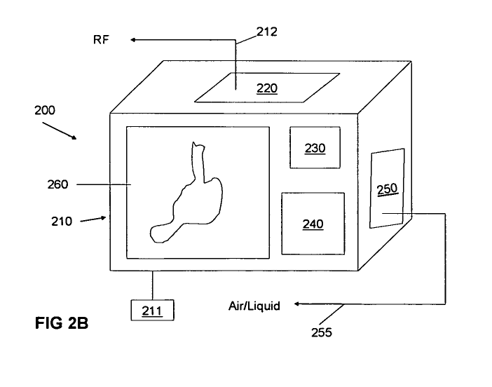

FIGS. 2A and 2B show portions of an exemplary apparatus 80 for treating body-weight related medical conditions.

Apparatus 80 includes a stomach treating portion 100 (FIG. 2A) and an external control portion 200 (FIG. 2B). Stomach treating portion 100 works inside the patient's digestive tract. External control portion 200 includes components for controlling, monitoring and viewing stomach treating portion 100.

As previously mentioned, stomach treating portion 100 works inside the patient's digestive tract. The distal end of stomach treating portion 100 includes a reference point positioner 110. Preferably, reference point positioner 110 comprises a positioning balloon 115 that can be inflated in the patient's body using a conventional air or liquid tube 116 that also acts as a catheter guide. Positioning balloon 115 is inflated after it passes through the pyloric sphincter 22 and seats against the distal side of the pyloric sphincter 22. Therefore, inflated positioning balloon 115 sets a reference point for tube 116 and allows proper positioning of the stomach treating portion 100 without using a visualization apparatus.

A stomach expander 120 may comprise, for example, a balloon assembly 130 integrated with a catheter 135 having a distal tip 135′. In FIG. 2A, balloon assembly 130 is collapsed. Catheter 135 allows balloon assembly 130 to be inserted into the patient's body over tube 116. Then, using an air line in handpiece 137 of catheter 135, balloon assembly 130 is inflated to expand the stomach's volume.

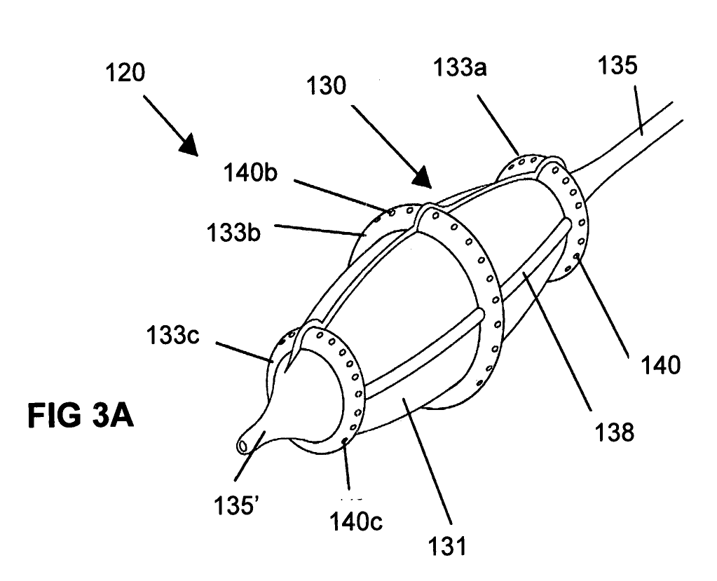

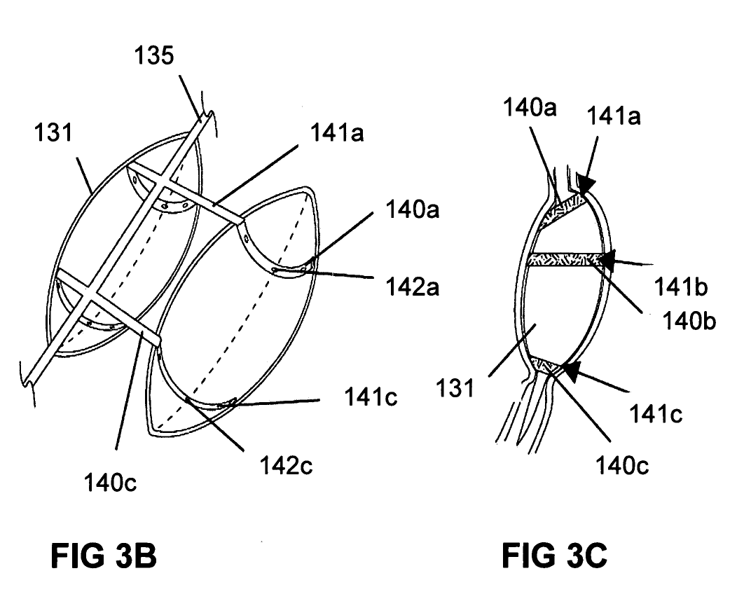

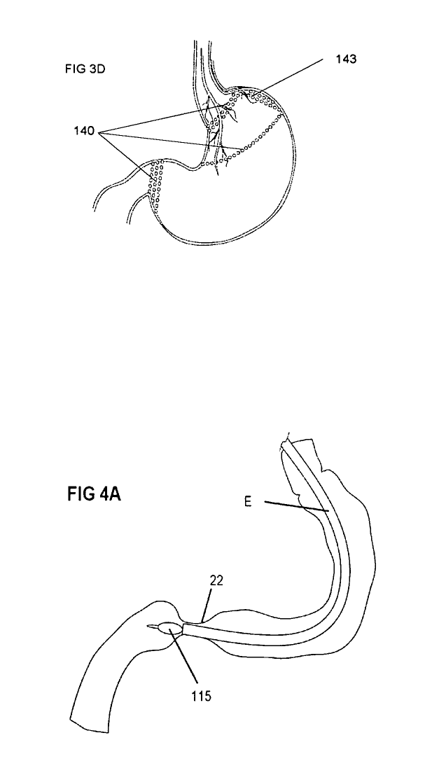

FIG. 3A-3D show various exemplary structures for stomach expander 120.

FIG. 3A shows an exemplary balloon assembly 130 including primary balloon 131 and secondary inflation rings 133a, 133b, 133c. Secondary inflation rings 133a, 133b, 133c circumferentially surround primary balloon 131 and are longitudinally positioned on the outer surface of primary balloon 131 at locations that will correspond to the vicinities of the cardiac, gastric/fundic, and pyloric zones when balloon assembly 130 is inflated inside the patient's stomach. Thus, a visualization apparatus is not needed for proper electrode positioning. Air channels 138 may interconnect secondary inflation rings 133a, 133b, 133c allowing a single air source to simultaneously inflate all the secondary inflation rings 133a, 133b, 133c.

Electrode groups 140a, 140b, 140c are circumferentially mounted to the outer circumference of secondary inflation rings 133a, 133b, 133c, respectively. Primary balloon 131 and secondary inflation rings 133 are independently and separately inflatable. Inflation of primary balloon 131 expands the stomach to stretch the pleated mucosa of the stomach and expose underlying nerves and stomach muscle. Inflation of secondary inflation rings 133a, 133b, and 133c assure a more accurate and complete surface contact between electrode groups 140a, 140b, 140c and treatment targets of the stomach.

In the exemplary balloon assemblies 130 shown in FIGS. 3B and 3C, there are no secondary inflation rings. Therefore, electrode groups 140a, 140b, 140c are attached along selected circumferences of primary balloon 131 that correspond to the vicinities of the fundus, peritoneum, and pyloris.

In FIG. 3B, there are two flexible circuits 141a, 141c associated with two electrode groups 140a, 140c, which are mounted to the inner surfaces of primary balloon 131. Accordingly, this shows that primary balloon 131 may have any number of electrode groups 140. Rivets 142a, 142c, piercing the primary balloon 131 surface, expose electrode groups 140a, 140c to the outside of balloon 131 so the electrodes can surface contact stomach tissue. In FIG. 3C, electrode groups 140a, 140b, 140c are made from flexible circuitry 141a, 141b, 141c etched onto the surface of primary balloon 131. Finally, FIG. 3D shows the electrode group contact points of an alternative stomach expander embodiment. In FIG. 3D, electrode groups 143 are provided for ablating branches of the any nerves connecting the stomach to the brain, for example, vagal nerve 15 connected to stomach 12. Therefore, the positioning of electrode groups 140, 143 on the primary balloon (not shown in FIG. 3D) corresponds to locations in close vicinity to branches of the vagal nerve 15.

Regardless of the configuration of balloon assembly 130, primary balloon 131 may be made from Mylar. Mylar restricts expansion of the primary balloon 131 within the stomach. Mylar, while expandable, is noncompliant. Therefore, a primary balloon 131 made of Mylar cannot infinitely expand and patient injury resulting from over-inflation of balloon assembly 130 can be reduced. When used for obesity treatment, balloon assembly 130 should be constructed so when inflated within the stomach, the stomach expands from its empty volume (about 1 liter) to at least about twice the stomach's empty volume (e.g. 2 liters). However, for other organs and other species, balloon assembly 130 may have different profiles or volumes. Stomach expansion stretches the pleated mucosa of the stomach and allows full surface contact between the electrode groups 140 and 143 and underlying stomach muscle and nerves. As shown in FIG. 3B, balloon 131 may comprise first and second balloon halves that are soft-welded together.

The individual electrodes of electrode groups 140, 143 are typically bifunctional, performing an energy emitting function and a sensing function. These types of sensors are extensively described in U.S. Pat. No. 6,872,206. Summarily, the energy emitting function is conducted by an energy emitting portion for heating and ablating tissue and which may comprise an RF energy emitter. The sensing function is conducted by a sensor portion that may include sensors or thermocouples, for measuring properties of the target region, such as temperature and impedance. As generally described below and extensively described in the '206 patent, measurement of these properties permits the use of feedback techniques to control delivery of the energy and administration of fluids for cooling and hydrating the targeted tissue.

To prevent overlapping ablation lesions, the electrode groups 140, 143 have enough electrodes to form all the desired lesions in a given target tissue with a single use (i.e., no repositioning of the balloon assembly 130 is required). Electrodes 140a, 140b, 140c are equally spaced about their respective circumferences of balloon assembly 130.

### External Control

FIG. 2A shows an external control portion 200 for apparatus 80 and including a control unit 210. Control unit 210 may include at least the following subassemblies: integrated RF generator 220, controller 230, I/O device 240, fluid delivery unit 250, and GUI 260. FIGS. 72A+ and associated text of U.S. Pat. No. 6,872,206 described this control unit in great detail. As described in the '206 patent, in alternative embodiments, the energy generator may deliver other forms of energy, such as heat, microwaves, infrared or visible laser energy to electrode groups 140a, 140b, 140c, 143. For brevity, we will not repeat the details of control unit 210 here.

Summarily, however, control unit 210 governs the power levels, cycles, and duration the radio frequency energy is transmitted through RF line 212 to electrode groups 140a, 140b, 140c, 143 to achieve and maintain power levels that achieve treatment objectives. Foot switch 211 allows hands-free control of energy emission. In tandem, control unit 210 controls delivery of processing fluid and, if needed, the removal of aspirated material through air and liquid lines 255.

The RF generator 220 of control unit 210 can include as many channels as necessary to supply treatment energy simultaneously to each electrode group 140a, 140b, 140c, 143.

Controller 210 includes an Input/Output (I/O) device 240. The I/O device 240 allows practioners to enter control and processing variables enabling control unit 210 to generate correct command signals. The I/O device 240 also receives real time processing feedback information from the one or more sensors associated with electrode groups 140a, 140b, 140c, 143, for processing by the controller 230, e.g., to govern energy application and processing fluid delivery. The I/O device 240 also includes a graphical user interface (GUI) 260 that graphically presents processing information to the practitioner for viewing or analysis.

### Therapeutic Procedure/Method

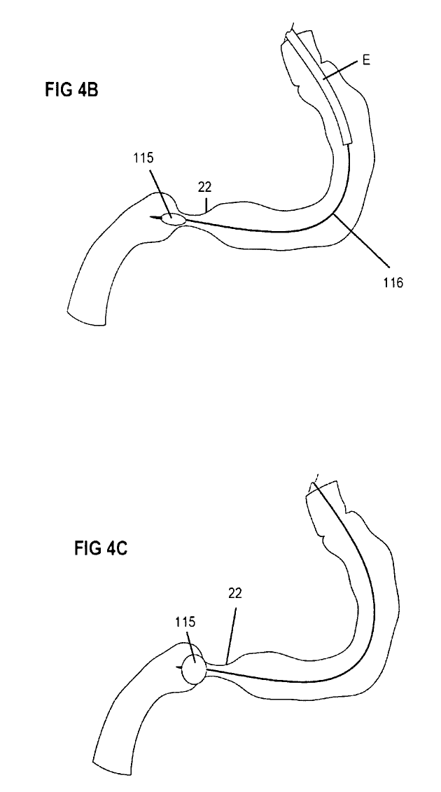

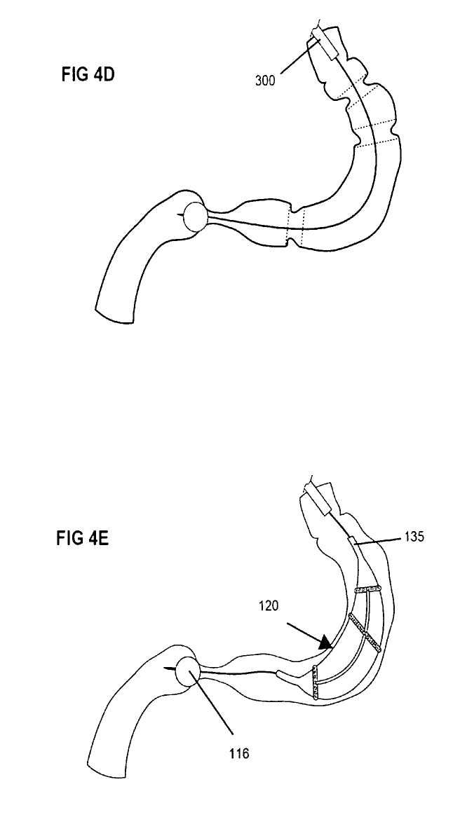

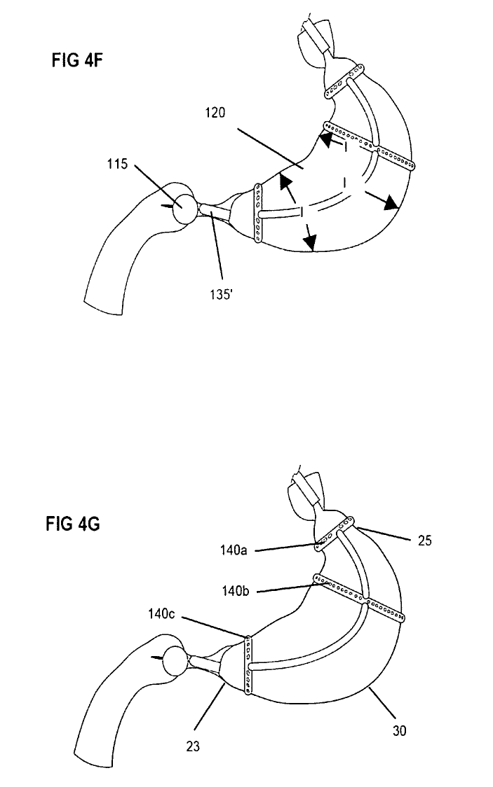

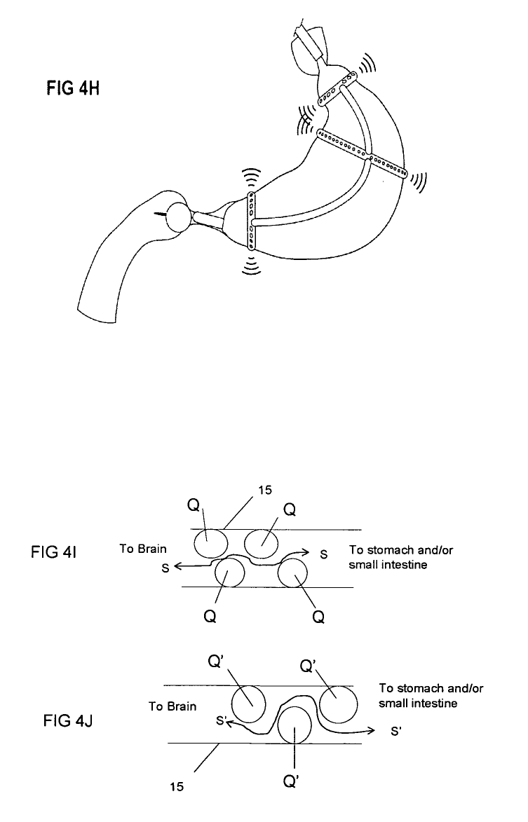

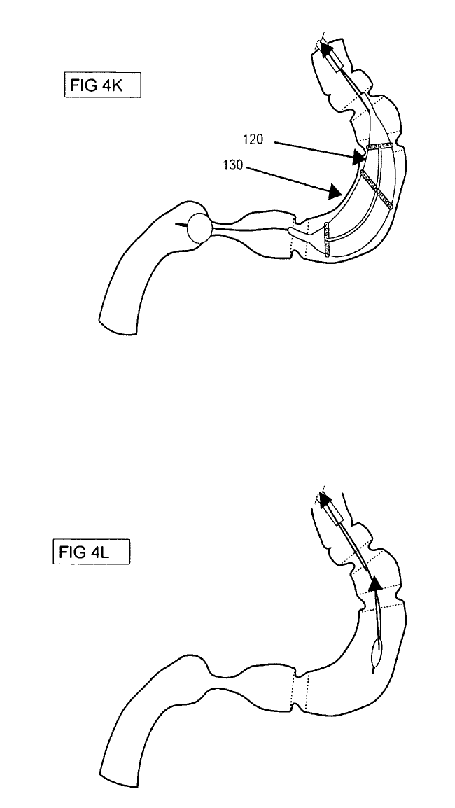

FIGS. 4A-L depict various steps of the therapeutic method. Patients can be treated outpatiently using conscious sedation. The procedure takes about one hour, including preparation and minimal recovery times. Because practioners need not make any incisions, the treatment is minimally invasive; in far contrast to the complex and highly invasive bariatric surgeries currently practiced. Practicing the disclosed process does not require the complete back-up of a hospital for emergencies, since the risk of serious problems during the treatment is low. Therefore, it may be possible to have treatment boutiques, such as in shopping malls, where the treatment can be carried out virtually “on demand” by trained practioners.

After patient sedation, endoscope E introduces the reference point positioner, assumed to be positioning balloon 115 for purposes of this description, into the patient's alimentary canal. The endoscope E forwards positioning balloon 115 through the stomach and onto the distal side of the pyloric sphincter 22 (FIG. 4A). The endoscope is retracted (FIG. 4B) and positioning balloon 115 inflated (FIG. 4C) to seal against the distal side of the pyloric sphincter 22. This sets a fixed reference point for tube 116.

A gastric introducer 300 positioned in the patient's throat (FIG. 4D), protects the esophageal walls during the next steps in the process.

Stomach expander 120 is now introduced into the patient's digestive system through the gastric introducer 300 and by catheter 135 riding over tube 116 (FIG. 4E). When distal tip 135′ of the catheter 135 contacts positioning balloon 115 and the closed pyloric sphincter (FIG. 4F), the practitioner stops inserting stomach expander 120 into the patient. Balloon assembly 130 is then inflated (FIG. 4F and inflation direction arrows I) until the stomach's volume becomes at least about twice its empty volume (e.g. to about 2 liters) (FIG. 4G). After inflation, electrode groups 140 and 143 automatically and directly contact vicinities of the nerves, muscles and glands of the treatment zones due to the positioning of electrode groups 140, 143 on balloon assembly 130.

The practitioner then, using control unit 210 and foot pedal 211 applies the selected energy source to these areas (FIG. 4H); energy may be in the form of RF, heat, microwaves, infrared or visible laser energy. For example, the practitioner activates the RF generator 220, resulting in the energy emitting portions of electrode groups 140a, 140b, 140c emitting energy to ablate the tissue in the treatment zones. During this time, using GUI 260 and feedback from the sensor portions of electrode groups 140a, 140b, 140c, the practitioner can watch for excessive temperatures. The duration of time and frequency of applied energy are, of course, responsive to judgments of medical personnel.

FIGS. 4I, J very schematically show the disruption and slowing of the travel of nerve pulses S, S′ between the stomach 12, the small intenstine, and the brain. In FIG. 4I, smaller ablated portions Q of exemplary nerve 15 disrupt the straight flow of nerve signal impulses S between the stomach, small intestine, and brain. In FIG. 4J, larger ablated portions Q′ of exemplary nerve 15 more greatly disrupt the straight flow of nerve signal impulses S′ between the stomach, small intestine, and brain. The size of ablated portions Q, Q′ and the desired degree of associated signal disruption are left to the sound judgment of the practioner after considering, for example, degree of patient obesity, strength of patient's hunger sensations, and variation in nerve size from patient to patient.

After the practioner is satisfied that the desired amount of tissue has been ablated and/or the pulse transmissions between nerves and the brain have been effected by the desired amount, energy application is stopped, the stomach expander 120 is deflated and balloon assembly 130 is withdrawn (FIG. 4K), as is the reference point positioner 110 and the gastro introducer 300 (FIG. 4L).

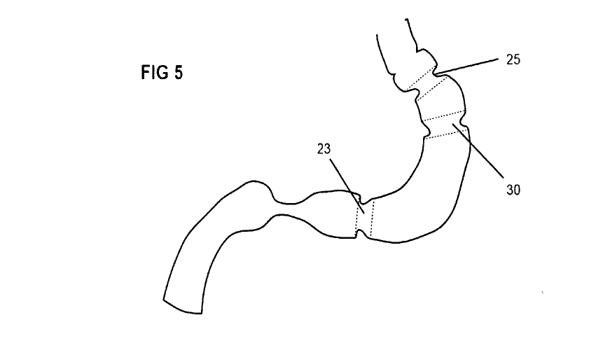

FIG. 5 depicts the muscle profile of a treated stomach about 3 months post-op. There are now major muscular constrictions and lesions (dead tissue) in the areas of the fundus 25, peritoneum 30 and pylorus 23. These muscular constrictions and associated lesions should cause patient weight loss for the reasons discussed above. Because the procedure does not cause complete cell death in the treated areas, over long periods of time continued healing may cause the stomach's muscle profile to return to normal. Accordingly, follow-up treatments may be required. However, due to the process' simplicity, this should not pose any undue risk or inconvenience to the patient.

### EXAMPLE

Currently, procedure testing has been conducted on rats, pigs, and Dunnarts, small marsupials about the size of a mouse. Dunnarts store about 25% of their total fat in their tail. Tail fat functions as an immediate source of energy supply. The amount of fat in the tail can be easily determined by measuring tail width. Accordingly, monitoring weight loss in Dunnarts is as simple as measuring tail width.

The test treatment included applying RF energy at approximately 460 hz to the gastric antrum of the Dunnart (pyloric zone) for about 1-3 minutes. This treatment created lesions in 25% of the treated Dunnart's gastric antrums. The SHAM (control) group received the same treatment as the treated Dunnart's, except for the application of energy to their antrum.

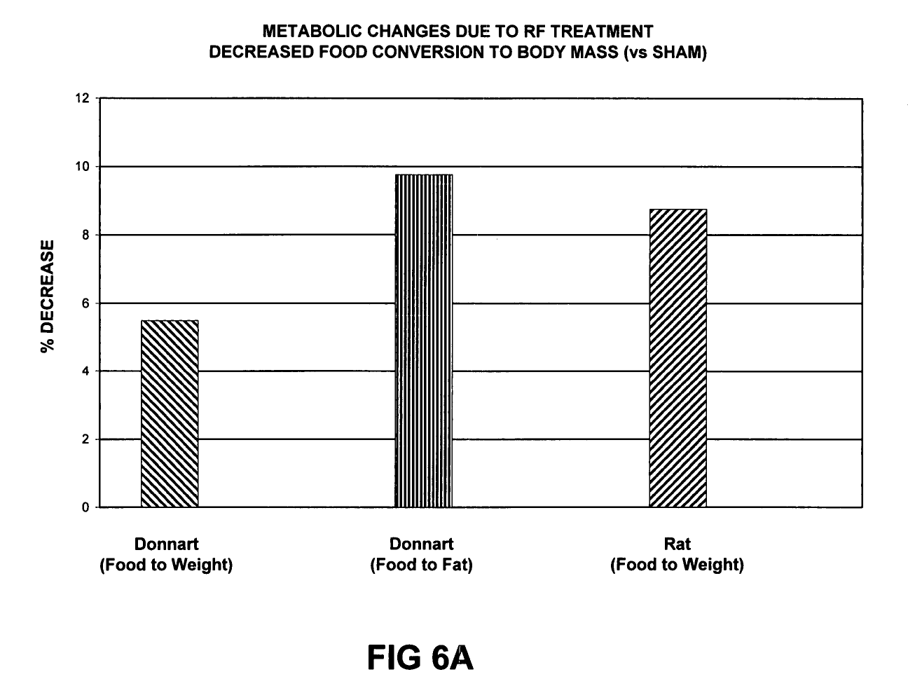

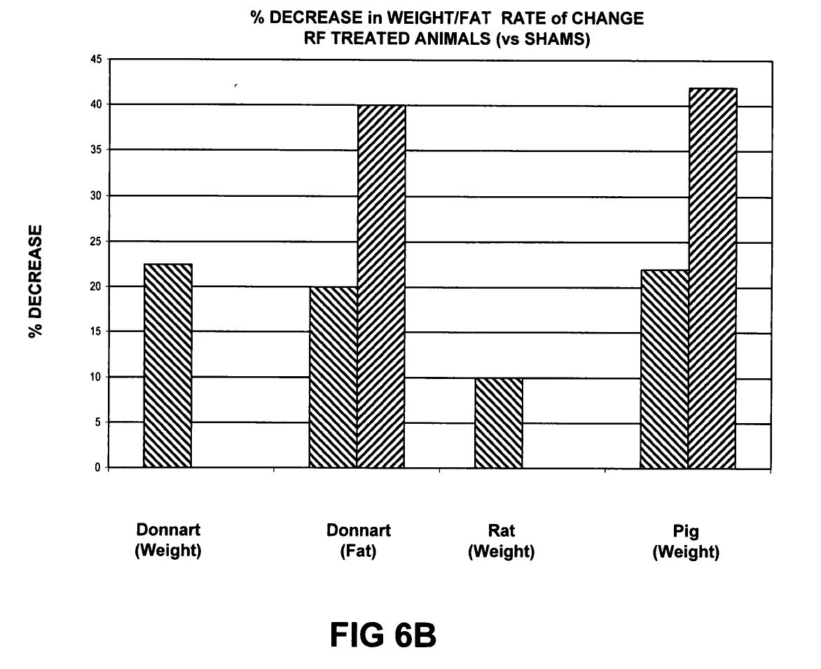

After 8-12 weeks, findings included:
Treated animals had lower weight increase rate on fatty diets than controls.
On standard diets, treated animals had 2% fat in their tails while controls had 25% fat in their tails.
Treated animals did not convert foodstuff to body weight as effectively as controls (see FIG. 6A).
The treated animals had no ill side affects from the treatment and exhibited normal eating, sleeping, feeding, and socializing behaviors within 2 days.

Test findings from rats, pigs, and Dunnarts are compared in FIG. 6B. Decreased weight results in all species and increases over time. Accordingly, it can be predicted that the treatment would produce significant weight loss in humans. FIG. 7 estimates human weight loss based on animal testing and in comparison to the conventional lap band treatment method. Summarily, it can be seen that the current procedure would produce significantly more weight loss than a conventional lap band method and with far less risks to the patient.

### Conclusion

While this application describes certain exemplary embodiments of treatments for weight-based medical conditions and apparatus useful for carry out the treatments, only the attached claims define the scope of the invention.

That which is claimed:

1. A treatment for medical conditions of hollow organs, comprising:
using at least one electrode positioned in the organ to directly apply energy to at least one surface of the organ to effect the organ's operation.

2. The treatment of claim 1, wherein the hollow organ is the stomach and the at least one surface of the stomach to which energy is directly applied is in the vicinity of at least a portion of a nerve communicating with the stomach and brain.

3. The treatment of claim 1, wherein the hollow organ is the stomach and the at least one surface of the stomach to which energy is directly applied is a stomach muscle surface.

4. The treatment of claim 2, further comprising also directly applying energy to at least one surface of the stomach's underlying glands to effect glandular emissions.

5. The treatment of claim 4, wherein the glandular emission is ghrelin.

6. A treatment for body-weight related medical conditions, comprising:
introducing an expandable element into a stomach;
expanding the stomach to expose the underlying nerves, muscles and glands of the stomach;
ablating at least one underlying nerve, muscle or gland of the stomach using a surface contact that applies energy directly to the nerve, muscle or gland.

7. The treatment of claim 6, wherein the at least one underlying muscle is in at least one of the cardiac, fundic/gastric, or pyloric zones.

8. The treatment of claim 7, further comprising treating underlying muscle in the cardiac, fundic/gastric, and pyloric zones.

9. The treatment of claim 6, wherein:
the expandable element comprises a balloon; and
further comprising associating energy transmitting electrodes with the surface of the balloon, whereby inserting and inflating the balloon in the stomach positions the transmitting electrodes in surface contact with at least one of the cardiac, fundic/gastric, or pyloric zones.

10. The treatment of claim 9, further comprising using a positioning element to assure that only selected areas of the stomach have their tissue ablated.

11. The treatment of claim 10, further comprising using a positioning element in the form of a second balloon inflated on the distal side of the pyloric sphincter.

12. An apparatus for treating medical conditions of hollow organs, comprising:
a catheter;
a hollow organ expander attached to the catheter and for expanding the organ to expose the hollow organ's underlying nerves and muscle;
at least one group of surface electrodes contacting the vicinity of at least one of the underlying nerve and muscle and emit energy away from the organ expander and towards the exposed underlying nerve and muscle.

13. The apparatus of claim 12, wherein the organ is the stomach, the organ expander is a stomach expander, and the at least one group of electrodes are in surface contact with the underlying nerve or muscle tissue of the stomach.

14. The apparatus of claim 13, wherein the stomach expander comprises a balloon inflatable from outside the body.

15. The apparatus of claim 14, wherein the balloon is shaped like the interior of the stomach and the at least one group of electrodes circumferentially surrounds the balloon in the area of at least one of the pyloric, gastric/fundic, and cardiac zones.

16. The apparatus of claim 15, wherein separate groups of electrodes surround the balloon in the areas of the pyloric, gastric/fundic, and cardiac zones.

17. The apparatus of claim 16, further comprising a reference point positioner for positioning a guide wire within the digestive system and the catheter rides along the guidewire until the electrodes correspond to the pyloric, gastric/fundic, and cardiac zones.

18. The apparatus of claim 14, wherein the balloon is made from mylar.
